(19) United States
(12) Patent Application Publication
(10) Pub. No.: US 2024/0269063 A1
(43) Pub. Date: Aug. 15, 2024

(54) Multi-Core Magnetic Metal Oxide Nanoparticles

(71) Applicant: UCL Business Ltd, London, Greater London (GB)

(72) Inventors: Liudmyla Storozhuk, London, Greater London (GB); Maximilian Bresenhard, London, Greater London (GB); Asterios Gavriilidis, London, Greater London (GB); Thanh Thi Kim Nguyen, London, Greater London (GB); Tung Duc Le, London, Greater London (GB)

(21) Appl. No.: 18/568,411

(22) PCT Filed: Jun. 8, 2022
(86) PCT No.: PCT/GB2022/051428

§ 371 Date: Dec. 8, 2023

(30) Foreign Application Priority Data
Jun. 9, 2021 (GB) 2108250.8

(51) Int. Cl. A61K 9/00; A61K 49/18; H01F 1/20

(52) U.S. Cl. CPC A61K 9/0009; A61K 49/183; H01F 1/20

(57) ABSTRACT

A method (100) for synthesising multi-core magnetic metal oxide nanoparticles is disclosed. The method comprises providing a first precursor mixture (102) comprising a first metal-containing precursor, a first solvent, and a first nanoparticle clustering agent, and heating the first precursor mixture to thermally decompose the first metal-containing precursor to produce a nanoparticle mixture (104) comprising multi-core magnetic metal oxide nanoparticles. The method further comprises performing at least one seeded growth step (106), each comprising a feeding step in which a further precursor mixture (108) is added to the nanoparticle mixture, the further precursor mixture comprising a further metal-containing precursor and a further solvent, and a heating step in which the nanoparticle mixture is heated to thermally decompose the second metal-containing precursor to achieve growth of the multi-core magnetic metal oxide nanoparticles.

### FIELD OF THE INVENTION

The invention relates to methods for synthesising multi-core magnetic metal oxide nanoparticles having an exceptionally high magnetic heating ability.

### BACKGROUND

The heat magnetic nanoparticles generate when exposed to an alternating magnetic field is exploited in many applications including catalysis, antimicrobial materials, and most notably magnetically induced hyperthermia (MIH) for cancer treatment. Due to the proven biocompatibility of iron-oxide nanoparticles (IONPs) they are the most commonly used magnetic nanoparticles for biomedical applications, with clinically approved applications in, for example, magnetic resonance imaging and drug/nutrient delivery.

MIH for cancer treatment relies on induced malignant cancer cell death after heating to temperatures above 40° C. The heating ability of the nanoparticles in an alternating magnetic field is therefore key to the success of this form of treatment. The more effectively the particles heat, the lower the concentration of nanoparticles required in the target tissue, which facilitates administration of the nanoparticles to the patient, reduces the chance of side effects, and improves the chances of achieving a sufficient concentration of nanoparticles in the target tissues to provide effective treatment.

The optimisation of magnetic nanoparticle properties for MIH is therefore an area of active research. The heating ability of the magnetic nanoparticles is considered the critical factor, and is usually prioritised when developing nanoparticle syntheses for MIH.

Clustered nanoparticles, sometimes called nanoclusters or nanoflowers, have been shown to have superior heating characteristics to their building blocks, i.e. the smaller single crystal cores from which they are comprised, which is thought to result from the magnetic interactions between individual cores in each cluster. Another appeal of clustered nanoparticles is that they remain superparamagnetic (which avoids further agglomeration and hence loss of colloidal stability) even when their dimension exceeds the superparamagnetic limit for individual (non-clustered or single-core) nanoparticles, which is around 25 nm (TEM diameter, D_{TEM}) for IONPs, for example.

Clustered metal oxide nanoparticles may be synthesised via thermal decomposition of a metal-containing precursor, such as a metal complex or a metal salt. Thermal decomposition synthesis of metal oxide nanoparticles such as IONPs is typically performed in a high boiling point solvent. Organic and polar solvents have been used in such syntheses. An advantage of using a polar solvent is that the resulting nanoparticles are hydrophilic due to the hydrophilic nature of the polar surface ligands provided by the polar solvent. Thermal decomposition syntheses may be enhanced by the addition of a reducing agent, which reduce the temperature at which thermal decomposition occurs. Oleylamine is a well-known example of a reducing agent used in thermal decomposition syntheses. In the polyol method, in which the polar solvent is a polyol, the polyol acts as a solvent and a reducing agent. Nanoparticles synthesised via the polyol method can be easily dispersed in aqueous media because the polyol molecules bind to the surface of the nanoparticles as hydrophilic ligands, which makes them amenable to further process steps that are performed in aqueous media and also makes them amenable to biological and medical applications.

There is still a need, however, for new methods of synthesising magnetic metal oxide nanoparticles that have an improved heating ability. In order to be practical and commercially viable, the new methods should be efficient, scalable, and capable of being performed in a relatively short time.

### SUMMARY OF THE INVENTION

According to a first aspect, the invention provides a method for synthesising multi-core magnetic metal oxide nanoparticles, the method comprising providing a first precursor mixture comprising a first metal-containing precursor, a first solvent, and a first nanoparticle clustering agent; heating the first precursor mixture to thermally decompose the first metal-containing precursor to produce a nanoparticle mixture comprising multi-core magnetic metal oxide nanoparticles; and performing a first seeded growth step comprising a feeding step in which a second precursor mixture is added to the nanoparticle mixture, the second precursor mixture comprising a second metal-containing precursor and a second solvent; and a heating step in which the nanoparticle mixture is heated to thermally decompose the second metal-containing precursor to achieve growth of the multi-core magnetic metal oxide nanoparticles.

The first solvent may be a first polar solvent, and/or the second solvent may be a second polar solvent.

The first precursor mixture may comprise a first reducing agent, and/or the second precursor mixture may comprise a second reducing agent. The second reducing agent may be the same as the first reducing agent.

The first solvent may also be the first reducing agent, and/or the second solvent may also be the second reducing agent.

The first solvent may be a first polyol solvent, and/or the second solvent may be a second polyol solvent.

The second solvent may be the same as the first solvent.

The second metal-containing precursor may be the same as the first metal-containing precursor.

The second precursor mixture may comprise a second nanoparticle clustering agent.

The second nanoparticle clustering agent may be the same as the first nanoparticle clustering agent.

The second precursor mixture may have the same composition as the first precursor mixture.

Prior to the first seeded growth step, the nanoparticle mixture may be cooled sufficiently to substantially halt growth of the multi-core magnetic metal oxide nanoparticles.

Following the heating step of the first seeded growth step, the nanoparticle mixture may be cooled sufficiently to substantially halt the growth of the multi-core magnetic metal oxide nanoparticles.

The method may further comprise performing at least one further (e.g. a second) seeded growth step after the first seeded growth step, each further seeded growth step comprising: a feeding step in which a further (e.g. third) precursor mixture is added to the nanoparticle mixture, the further precursor mixture comprising a further metal-containing precursor and a further solvent; and a heating step in which the nanoparticle mixture is heated to thermally decompose the further metal-containing precursor to achieve further growth of the multi-core magnetic metal oxide nanoparticles.

In each further seeded growth step, the further precursor mixture may comprise a further reducing agent.

In each further seeded growth step, the further reducing agent may be the same as the first reducing agent and/or the second reducing agent.

In each further seeded growth step, the further solvent may also be the further reducing agent.

In each further seeded growth step, the further solvent may be a further polyol solvent.

More than one further seeded growth step may be performed, and the further precursor mixtures in each further seeded growth step may have the same composition.

The further metal-containing precursor in each further seeded growth step may be the same as the first metal-containing precursor and/or the second metal-containing precursor.

The further solvent in each further seeded growth step may be the same as the first solvent and/or the second solvent.

Each further precursor mixture may comprise a further nanoparticle clustering agent.

The further nanoparticle clustering agent in each further seeded growth step may be the same as the first and/or second nanoparticle clustering agent.

The further precursor mixture in each further seeded growth step may have the same composition as the first precursor mixture and/or the second precursor mixture.

In each further seeded growth step, following the heating step, the nanoparticle mixture may be cooled sufficiently to substantially halt the growth of the multi-core magnetic metal oxide nanoparticles.

The nanoparticle mixture may be cooled sufficiently to halt growth of the nanoparticles/substantially terminate thermal decomposition of the metal-containing precursor before the first seeded growth step and after each of the seeded growth steps.

The nanoparticle clustering agent in any or each of the precursor mixtures may be a polyelectrolyte.

The nanoparticle clustering agent in any or each of the precursor mixtures may be polyacrylic acid.

In the first precursor mixture, the molar ratio of the first nanoparticle clustering agent to the metal provided by the first metal-containing precursor may be in the range of 0.6:1 to 1.4:1. For example, the molar ratio of the first nanoparticle clustering agent to the first metal-containing precursor may be in the range of 0.6:1 to 1.4:1.

In each of the precursor mixtures, the molar ratio of the respective nanoparticle clustering agent to the metal provided by the respective metal-containing precursor may be in the range of 0.6:1 to 1.4:1. For example, the molar ratio of the respective nanoparticle clustering agent to the respective metal-containing precursor may be in the range of 0.6:1 to 1.4:1.

The metal-containing precursor in any or each of the precursor mixtures may be a metal salt or a metal complex.

The multi-core magnetic metal oxide nanoparticles may be ferrite multi-core magnetic metal oxide nanoparticles. The metal-containing precursor in each of the precursor mixtures may be an iron-containing precursor. The metal-containing precursor in any or each of the precursor mixtures may be iron(III) acetylacetonate or iron(III) acetate.

Each of the precursor mixtures may further comprises a respective dopant-containing precursor. The ferrite multi-core magnetic metal oxide nanoparticles may be doped ferrite multi-core magnetic metal oxide nanoparticles comprising the dopant(s).

The dopant in each of the dopant-containing precursors may be a transition metal.

The dopant in each of the dopant-containing precursors may be copper or zinc.

Each of the dopant-containing precursors may be a metal acetylacetonate, or a metal acetate, the metal being the dopant.

Each of the dopant-containing precursors may be copper (II) acetylacetonate, copper (II) acetate, zinc (II) acetylacetonate or zinc (II) acetate.

Each of the respective dopant-containing precursors may be the same dopant-containing precursor, i.e. the same substance.

The amount by weight of the dopant present in the precursor mixture may be less than 10% of the combined amount by weight of iron and the dopant present in the precursor mixture.

The multi-core magnetic oxide nanoparticles that are synthesised by the method may comprise the dopant(s) in a total amount of less than 20% by weight of the total weight of the multi-core magnetic oxide nanoparticles.

The polyol solvent in each of the precursor mixtures may be triethylene glycol or tetraethylene glycol.

The first precursor mixture may be heated to a temperature sufficient to decompose the first metal-containing precursor to form the multi-core magnetic metal oxide nanoparticles for a period of at least about 20 minutes, optionally at least about 30 minutes.

In the heating step of each seeded growth step, the nanoparticle mixture may be heated sufficiently to decompose the metal-containing precursor to form the multi-core magnetic metal oxide nanoparticles for a period of at least about 20 minutes, optionally at least about 30 minutes.

The method may further comprise a ligand-exchange step in which solvent ligands (i.e. ligands provided by the solvent) bound to the surface of the nanoparticles are replaced or exchanged with stabilising ligands that enhance the colloidal stability of the nanoparticles in aqueous media.

The synthesised multi-core magnetic metal oxide nanoparticles may have an intrinsic loss parameter of at least about 7 nH m² kg_{M}⁻¹, preferably at least about 8 nH m² kg_{M}⁻¹, where kg_{M} is the weight in kg of the metal component of the metal oxide of the magnetic metal oxide nanoparticles.

The synthesised multi-core magnetic metal oxide nanoparticles may be superparamagnetic.

The synthesised multi-core magnetic metal oxide nanoparticles may have an average diameter of at least about 25 nm, as measured by transmission electron microscopy. The synthesised multi-core magnetic metal oxide nanoparticles may have individual core diameters of less than about 15 nm, as measured by X-ray diffraction.

The synthesised multi-core magnetic metal oxide nanoparticles may comprise monocrystalline individual cores. The synthesised multi-core magnetic metal oxide nanoparticles may comprise substantially aligned monocrystalline individual cores.

The synthesised multi-core magnetic metal oxide nanoparticles may be nanoflowers.

The method for synthesising multi-core magnetic metal oxide nanoparticles may comprise providing a first precursor mixture comprising a (first amount of a) metal-containing precursor, a (first amount of a) first solvent, and a (first amount of a) first nanoparticle clustering agent; heating the first precursor mixture to thermally decompose the first metal-containing precursor to produce a nanoparticle mixture comprising multi-core magnetic metal oxide nanoparticles; and performing a first seeded growth step comprising a feeding step in which a second precursor mixture is added to the nanoparticle mixture, the second precursor mixture comprising (a second amount of) the metal-containing precursor, (a second amount of) the solvent and optionally (a second amount of) the nanoparticle clustering agent; and a heating step in which the nanoparticle mixture is heated to thermally decompose the metal-containing precursor to achieve growth of the multi-core magnetic metal oxide nanoparticles. The further precursor mixture(s) may comprise (a third amount of) the metal-containing precursor, (a third amount of) the solvent and optionally (a third amount of) the nanoparticle clustering agent. The first, second, and third amounts of the various components may be the same or different in each of the precursor mixtures.

The method for synthesising multi-core magnetic metal oxide nanoparticles may comprise providing a first amount of a precursor mixture comprising a metal-containing precursor, a first solvent, and a first nanoparticle clustering agent; heating the first precursor mixture to thermally decompose the first metal-containing precursor to produce a nanoparticle mixture comprising multi-core magnetic metal oxide nanoparticles; and performing a first seeded growth step comprising a feeding step in which a second amount of the precursor mixture is added to the nanoparticle mixture; and a heating step in which the nanoparticle mixture is heated to thermally decompose the metal-containing precursor to achieve growth of the multi-core magnetic metal oxide nanoparticles. The method may further comprise performing at least one further (e.g. a second) seeded growth step after the first seeded growth step, each further seeded growth step comprising: a feeding step in which a further (e.g. third) amount of the precursor mixture is added to the nanoparticle mixture; and a heating step in which the nanoparticle mixture is heated to thermally decompose the metal-containing precursor to achieve further growth of the multi-core magnetic metal oxide nanoparticles. The first, second, and further (e.g. third) amounts may be the same or different.

The invention also provides a composition (e.g. a solution or dispersion) comprising multi-core magnetic metal oxide nanoparticles synthesised according to the method of the invention.

The invention also provides multi-core magnetic metal oxide nanoparticles synthesised according to the method of the invention.

The multi-core magnetic oxide nanoparticles may comprise one or more dopants.

The multi-core magnetic oxide nanoparticles may comprise one or more dopants in a total combined amount of less than 20% by weight.

The one or more dopants may comprise or may be copper and/or zinc.

The invention also provides a composition (e.g. a solution or dispersion) comprising multi-core magnetic metal oxide nanoparticles, the magnetic metal oxide nanoparticles having an intrinsic loss power of at least about 6 nH m² kg_{M}⁻¹, or at least about 7 nH m² kg_{M}⁻¹.

The invention also provides multi-core magnetic metal oxide nanoparticles having an intrinsic loss power of at least about 6 nH m² kg_{M}⁻¹, or at least about 7 nH m² kg_{M}⁻¹.

The compositions and nanoparticles of the invention may be for use in magnetic hyperthermia therapy. For example, the compositions may be for use in the treatment of cancer by magnetic hyperthermia therapy.

The present specification also discloses use of the multi-core magnetic metal oxide nanoparticles of the invention in magnetic heating, for example for use in MIH therapy. The MIH therapy may be for treating cancer. The present specification also discloses use of the multi-core magnetic metal oxide nanoparticles of the invention in magnetic particle imaging (MPI). The multi-core magnetic metal oxide nanoparticles of the invention may be used in MIH therapy in combination with magnetic particle imaging (MPI).

The present specification also discloses a method for treating cancer by MIH therapy using multi-core magnetic metal oxide nanoparticles according to the invention, for example synthesised according to the method of the invention or as otherwise defined herein.

### BRIEF DESCRIPTION OF FIGURES

The invention will now be described by way of example only with reference to the accompanying figures, in which:

FIG. 1 shows a diagram schematically illustrating a seeded growth method in accordance with the invention.

FIG. 2 shows data summarising the properties of the nanoparticles synthesised in the examples. Table 1 summarises the properties of the nanoparticles synthesised in Reference Example 1, Table 2 summarises the properties of the nanoparticles synthesised in Reference Example 2, and Table 3 summarises the properties of the nanoparticles synthesised in Example 1.

FIG. 3a shows transmission electron microscopy (TEM) images of nanoparticles synthesised in Reference Example 1. The panels show, clockwise from top left, nanoparticles synthesised with a reaction time of 30 minutes, 1 hour, 24 hours, and 6 hours.

FIG. 3b shows TEM images of nanoparticles synthesised in Reference Example 2. The panels show, clockwise from top left, nanoparticles synthesised with a polyacrylic acid (PAA) concentration of 20 mM, 33 mM, 100 mM, and 66 mM.

FIG. 4 shows TEM images of nanoparticles synthesised in Example 1. The left-hand panel shows nanoparticles following a first seeded growth step and the right-hand panel shows nanoparticles following a second seeded growth step.

FIG. 5 shows aberration corrected transmission electron micrograph of the nanoparticle seeds (left) of Example 1 and after the second seeded growth step (right) of Example 1. The superimposed lines show the low index {111} facets. The insets show the FFT taken from the dashed white box, with the iron oxide viewed down the [310] zone axis in the left-hand (seed) TEM image, and [110] zone axis in the right-hand (second seeded growth) panel. The white lines in the FFT show the angle of variation in the FFT spot.

FIG. 6 shows an FTIR spectra of IONPs synthesised with 66 mM PAA after exchanging the polyol ligands with sodium tripolyphosphate (STPP) in Example 2.

FIG. 7 shows the time-dependence hydrodynamic diameter evolution of the IONP nanoclusters following the ligand exchange step of Example 2.

FIG. 8a shows TEM images of nanoparticles synthesised in Example 3a. The left-hand panel shows the copper-doped IONP seeds and the right-hand panel shows nanoparticles following the second seeded growth step.

FIG. 8b shows TEM images of nanoparticles synthesised in Example 3b. The left-hand panel shows the zinc-doped IONP seeds and the right-hand panel shows nanoparticles following the second seeded growth step.

FIG. 9a shows elemental mapping TEM images of the copper-doped IONP seeds synthesised in Example 3a.

FIG. 9b shows elemental mapping TEM images of the copper-doped IONP seeds synthesised in Example 3b.

### DETAILED DESCRIPTION

The inventors have discovered that magnetic metal oxide nanoparticles having an exceptionally high heating ability may be synthesised using a seeded-growth thermal decomposition method. The seeded growth approach surprisingly results in far higher heating abilities than would otherwise be expected based on the size of the resulting nanoparticles. The resulting multi-core nanoparticles may be interchangeably referred to as nanoclusters, clustered nanoparticles, or nanoflowers. It is thought that the enhanced heating abilities result from the novel structural properties of the resulting clustered nanoparticles. For example, although the single crystal cores of the multi-core nanoclusters are substantially aligned, their alignment is not perfect and increases with each seeded growth step. This implies the presence of an increased number of defects in the crystals that form the individual cores of the multi-core nanoparticles, which is thought to contribute to the increased heating ability of nanoparticles synthesised using the seeded growth method compared to those synthesised using a single-step thermal decomposition synthesis.

Advantageously, nanoclusters synthesised by the seeded growth method have been shown to be superparamagnetic and readily dispersible in water or other aqueous media. The colloidal stability of the nanoclusters may also be increased further following a ligand exchange process in which the surface ligands of the nanoclusters provided by the solvent in which the synthesis is performed are replaced by charged (ionic) ligands with a high hydrophilicity.

In the seeded growth method of the invention, a precursor mixture (e.g. a solution or dispersion) comprising a metal-containing precursor and a nanoparticle clustering agent is heated to yield a nanoparticle seed mixture comprising seed multi-core magnetic metal oxide nanoparticles via thermal decomposition of the metal precursor. Following this, one or more seeded growth steps are performed, in which further amounts of precursor mixture are added to the nanoparticle seed mixture in a feeding step, and the resulting reaction mixture (i.e. the nanoparticle seed mixture plus added precursor mixture) is then heated again to achieve seeded growth of the multi-core magnetic metal oxide nanoparticles via thermal decomposition of the metal precursor added in the feeding step. In each seeded growth step the nanoclusters increase in size as more single-crystal cores, or grains as they are sometimes known, are added to the nanoclusters. This means that the overall size of the nanoclusters increases with each seeded growth step, while the individual grain size remains relatively constant.

The resulting nanoparticles retain their superparamagnetic properties due to the size of the individual grains remaining below the superparamagnetic limit, even though the overall size of the nanoclusters themselves is much larger. This avoids further agglomeration, and hence precipitation, of the nanoparticles following their formation.

A further advantage of the seeded growth method is that each seeded growth step, and the initial step of forming the seed mixture, is performed in a relatively short period of time. This makes the method practical, scalable, and commercially viable.

The metal oxide nanoparticles synthesised in the method of the invention are magnetic metal oxide nanoparticles such as ferrite (i.e. iron oxide) nanoparticles. The nanoparticles may therefore comprise, consist of, or consist essentially of at least one magnetic metal oxide, such as at least one iron oxide in the case of iron oxide nanoparticles. There are many different iron oxides, such as magnetite (Fe₃O₄), maghemite (γ-Fe₂O₃), and other Fe(II) and Fe(III) oxides, and iron oxide nanoparticles comprise one or more of such iron oxides. The magnetic metal oxide nanoparticles may also comprise other metals, elements, or compounds in addition to the magnetic metal oxides. For example, metal oxide nanoparticles may also comprise one or more dopants, such as nickel, cobalt, copper, zinc, manganese, or magnesium. For example, IONPs may be nickel-, cobalt-, copper-, zinc- or manganese-doped IONPs, or they may be doped with other metals. In particular, the iron oxide nanoparticles may be doped with other ferromagnetic metals, such as cobalt and/or nickel. The IONPs may therefore be cobalt- and/or nickel-doped IONPs, or the dopant may be another metal, such as a transition metal. The inventors have discovered that IONPs doped with copper and/or zinc exhibit exceptionally good magnetic heating abilities. As such, the IONPs may in particular be copper and/or zinc-doped IONPs, as is explained in further detail below.

The total amount (by weight) of the dopants in the IONPs is less than the amount of the iron oxides in the IONPs, typically substantially less. For example, IONPs typically comprise at least 80% by weight (wt./wt.) iron oxides, more typically at least 90% wt./wt. iron oxides. Put another way, the IONPs may comprise less than 20% by weight dopants, for example less than 10% by weight dopants. In other words, the IONPs that are synthesised by the method of the invention may comprise the dopants in a total amount by weight (i.e. the total combined amount by weight of the dopants relative to the total weight of the IONPs) of less than 20%, or less than 10%.

Where a specific magnetic metal oxide or type of magnetic metal oxide nanoparticle is referred to in relation to the invention, such as iron oxide or IONPs, it should be understood that other suitable magnetic metal oxides or magnetic metal oxide nanoparticles could be used instead.

The magnetic metal oxide nanoparticles comprise, consist of, or consist essentially of magnetic metal oxide phases, such as spinel structure metal oxide phases. Where spinel structure phases are referred to herein, this should be understood to include inverse spinel structures. Iron oxide nanoparticles produced by the method of the invention may therefore comprise, consist of, or consist essentially of magnetite and/or maghemite.

### Nanocluster Seed Mixture Formation

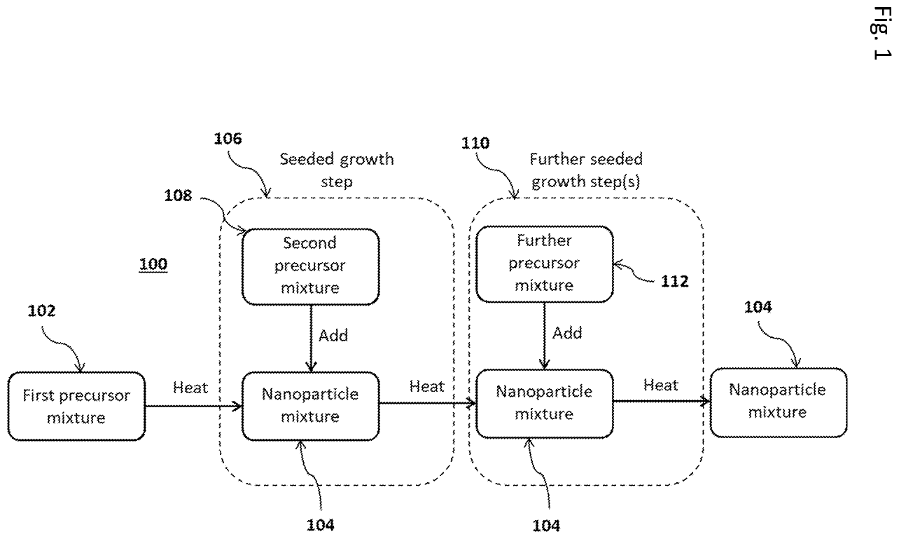

Referring to FIG. 1, a seeded growth method 100 according to the invention includes providing a first precursor mixture 102. The first precursor mixture comprises a first metal precursor and a first nanoparticle clustering agent. The first precursor mixture also comprises a solvent, in which the metal precursor and the nanoparticle clustering agent are solvated or otherwise dispersed. The first precursor mixture may therefore be referred to as a solution or, more generally, a dispersion. Due to the size of nanoparticles, nanoparticle solutions are sometimes referred to as colloidal solutions, and the terms “solution” and “dispersion” are intended to encompass such colloidal solutions. In general, where the term “mixture” is used herein, it may be understood to mean solution or dispersion, and this includes colloidal dispersions or solutions of nanoparticles. Nanoparticles synthesised by the method of the invention are small enough, having a hydrodynamic diameter of less than 150 nm (as determined by dynamic light scattering (DLS)), that they are colloidally stable in aqueous media and therefore form a solution in aqueous media.

The metal precursor comprises the metal, typically in the form of metal ions, of which the synthesised nanoparticles are comprised. In other words, the metal precursor contains the same metal as the synthesised metal oxide nanoparticles such that the metal of the metal precursor is the same as the metal of the metal oxide of the nanoparticles. For example, if iron oxide nanoparticles are to be synthesised, the metal-containing precursor comprises iron in the form of iron ions, e.g. ferrous (Fe²⁺) and/or ferric (Fe³⁺) ions. In general, the metal of the metal precursor is a magnetic metal, more specifically a ferromagnetic metal. The metal precursor may comprise a metal salt or metal complex, such as an iron salt or an iron complex. When IONPs are synthesised, the metal precursor may be or may comprise iron(III) acetylacetonate (Fe(acac)₃). Other suitable iron-containing precursors include iron chlorides (FeClₓ) and iron acetates (Fe(ac)ₓ), for example. The metal precursor supplies the metal ions for the synthesis of the metal oxide nanoparticles. The first precursor solution therefore comprises metal ions, e.g. Fe ions, provided by the metal precursor.

The first nanoparticle clustering agent promotes the clustering of individual nanoparticles to form multi-core nanoparticles, or nanoclusters. The nanoclusters comprise multiple cores, or grains, which are typically single crystal, or monocrystalline, nanostructures. These individual cores are initially formed as separate nanoparticles, and the nanoparticle clustering agent promotes the clustering of these individual nanoparticles to form multi-core nanoclusters. The nanoparticle clustering agent typically acts to bind the individual nanoparticles together, typically due to molecules of the clustering agent binding with the surface of multiple nanoparticles, thus bringing them together. The nanoparticle clustering agent may therefore be otherwise known as a nanoparticle binder or nanoparticle binding agent.

The nanoparticle clustering agent may be a polyelectrolyte, such as a polyanionic polymer. The many electrolyte groups present along the length of polyelectrolyte chains allow the polyelectrolyte molecules to effectively bind with multiple individual cores and thus cause clustering. The clustering agent preferably comprises PAA. When thermal decomposition is performed in the presence of a nanoparticle clustering agent, such as PAA, clustering of the individual single-crystal nanoparticle cores is enhanced, resulting in the preferential formation of multi-core nanoparticles, otherwise known as nanoclusters.

In the first precursor solution, the molar ratio of the first nanoparticle clustering agent to the metal (e.g. metal ions) provided by the first metal precursor may be at least about 0.2:1, preferably at least about 0.3:1, more preferably at least about 0.6:1, more preferably still at least about 0.8:1. For lower ratios of nanoparticle clustering agent to metal the effect of the clustering agent is less pronounced and clustering is not seen to be so effectively promoted, resulting in smaller nanoclusters and lower heating abilities of the resulting nanoparticles. At the upper end, the molar ratio of the nanoparticle clustering agent to the metal in the first precursor solution may be less than about 1.8:1, preferably less than 1.6:1, more preferably less than 1.4:1, more preferably still less than about 1.2:1. At higher molar ratios the solution becomes increasingly viscous due to higher amounts of PAA in the solution. However, this effect can be mitigated against by reducing the absolute concentrations of both PAA and the metal precursor. For example, the molar ratio may be in the range of about 0.6:1 to about 1.4:1, more preferably from about 0.8:1 to about 1.2:1. A particularly preferably ratio is about 1:1. Surprisingly, it has been found that the within these ranges the properties of the resulting nanocluster solutions and the heating abilities of the nanoclusters are improved, particularly when the nanoparticle clustering agent is PAA. For example, the ratios above may refer to the molar ratio of PAA to iron (e.g. iron ions) in the first precursor mixture, and may therefore be the ratio of PAA to the iron-containing precursor, e.g. PAA to iron(III) acetylacetonate, in the first precursor mixture.

The first precursor solution may comprise a reducing agent, such as oleylamine or a polyol. Reducing agents reduce the temperature at which thermal decomposition occurs, thus allowing the thermal decomposition synthesis to be performed at lower temperatures.

The solvent is preferably a high boiling point solvent. For example, the solvent may have a boiling point higher than the temperature at which thermal decomposition of the metal precursor to form metal oxide nanoparticles occurs. Therefore, the solvent may have a boiling point that is higher than the temperature to which the precursor mixture (and the seed/nanoparticle mixture in the subsequent seeded growth steps) is heated in the heating steps of the method. For example, the solvent may have a boiling point above 280° C.

The first solvent is preferably a polar solvent. An advantage of using a polar solvent is that the resulting nanoparticles are dispersible in water or aqueous media due to the hydrophilic nature of the polar surface ligands provided by the polar solvent. In particular, the solvent may comprise a polyol. The polyol acts as both the solvent and the reducing agent. Nanoparticles synthesised via the polyol method can be easily dispersed in aqueous media and other polar solvents because the polyol molecules bind to the surface of the nanoparticles as hydrophilic ligands, which makes them amenable to biological and medical applications without further surface modification. The polyol molecules can also be readily exchanged in a ligand exchange step following the synthesis of the nanoparticles to enhance the water solubility and colloidal stability of the nanoclusters, which makes them more amenable to biological and medical applications. The polyol solvent may, for example, comprise triethylene glycol (TREG) and/or tetraethylene glycol (TEG).

If synthesising IONPs, the metal precursor may be an iron salt or complex such as iron(III) acetylacetonate (Fe(acac)₃), the nanoparticle clustering agent may be a polyelectrolyte such as PAA, and the solvent may be a polyol solvent, comprising at least one polyol such as TREG or TEG. However, other combinations are possible.

The first precursor mixture is then heated to produce a nanoparticle mixture 104, or seed mixture, comprising multi-core magnetic metal oxide nanoparticles via thermal decomposition of the metal precursor. The first precursor mixture is therefore heated to an elevated temperature sufficient to decompose the metal precursor to form the multi-core magnetic metal oxide nanoparticles. The precursor mixture may, for example, be heated to a temperature of at least about 280° C. This elevated temperature may be referred to as the first reaction temperature. The precursor mixture may be heated to at least the first reaction temperature, for example held at the first reaction temperature, for a certain period of time, known as the first reaction time. The first reaction time may be at least about 20 minutes, or at least about 30 minutes. For example, the first reaction time may be about 30 minutes. The precursor mixture may be heated to the first reaction temperature in a two-step process comprising a first heating step of heating to a temperature sufficient to substantially dissolve the metal precursor and nanoparticle clustering agent in the solvent (e.g. about 180° C.), a holding step, in which the temperature is held at this temperature for a time sufficient to substantially dissolve the metal precursor and nanoparticle clustering agent (e.g. about 30 mins), and then a second heating step, in which the temperature of the precursor mixture is raised to the reaction temperature.

Following the thermal decomposition step, the resulting nanoparticle solution is cooled sufficiently to substantially prevent further thermal decomposition of the metal precursor and to substantially halt growth of the nanoparticles. The temperature of the nanoparticle solution is therefore reduced to a temperature insufficient to cause thermal decomposition of the metal precursor. In other words, the temperature of the nanoparticle solution is reduced sufficiently to quench growth of the nanoparticles or to substantially prevent further growth of the nanoparticles. The nanoparticle solution may, for example, be reduced to ambient (e.g. room) temperature, such as to about 25° C.

### Seeded Growth Steps

The method then involves performing one or more seeded growth steps 106, 110 in which the nanoclusters are grown in size in a stepwise manner. Each seeded growth step comprises a feeding step and a heating step. The method involves at least a first seeded growth step, and may comprise one or more further seeded growth steps following the first seeded growth step. For example, the method may comprise a first seeded growth step and a second seeded growth step.

In the feeding step of the first seeded growth step a second precursor mixture 108 (e.g. a second precursor solution) is added to the nanoparticle or seed mixture. The second precursor mixture comprises a second metal precursor and a second solvent. The second precursor mixture may also comprise a second nanoparticle clustering agent and/or a second reducing agent. However, it is not necessary for these additional components to be present, and the amount of nanoparticle clustering agent remaining from the first precursor solution may be sufficient to achieve clustering of the nanoparticles in the first seeded growth step without the need to add further nanoparticle clustering agent in the second precursor solution.

The components of the second precursor mixture may be as described above in relation to the equivalent components in the first precursor mixture. For example, the second solvent may be a polyol solvent, the second metal precursor may be iron(III) acetylacetonate, and the second nanoparticle clustering agent, if present, may be PAA. The corresponding components of the first and second precursor mixtures may be the same. For example, the first and second metal-containing precursors may be the same, the first and second solvents may be the same, and the first and second nanoparticle clustering agents may be the same. In such cases, it may be said that the first precursor mixture comprises first amounts of a metal-containing precursor/solvent/nanoparticle clustering agent and that the second precursor mixture comprises second amounts of the metal-containing precursor/solvent/nanoparticle clustering agent. Alternatively, it may be said that the first precursor mixture comprises a first precursor/solvent/nanoparticle clustering agent and that the second precursor mixture comprises the (same) metal-containing precursor/solvent/nanoparticle clustering agent. The second precursor mixture may, for example, have the same composition as the first precursor mixture, such that the chemical identity of each of the components is the same, and the relative amounts of each of the components in the second precursor mixture are the same as in the first precursor mixture. If this is the case, it could then be said that the first precursor mixture is a first amount of a precursor mixture, and that the second precursor mixture is a second amount of the precursor mixture. Likewise, the further precursor mixtures added in the subsequent seeded growth steps may be referred to as further amounts of the precursor mixture if these have the same composition as the first precursor mixture. It is not necessary for the second precursor mixture to have the same composition as the first precursor mixture, but typically the chemical identities of the components in the precursor mixtures will be the same. For example, the second metal precursor will typically be the same as the first metal precursor (i.e. first and second metal precursors will be the same compound, such as iron(III) acetylacetonate), the second solvent will typically be the same as the first solvent, and, if present, the second nanoparticle clustering agent will typically be the same as the first nanoparticle clustering agent. If a separate reducing agent is present, the second reducing agent will typically be the same as the first reducing agent. In general, the second precursor solution may be prepared as described above in relation to the first precursor solution.

In the second precursor solution, the molar ratio of the second nanoparticle clustering agent to the metal (e.g. metal ions) provided by the second metal precursor may be as described above in relation to the first precursor solution. For example, the molar ratio may be in the range of about 0.6:1 to 1.4:1, more preferably 0.8:1 to 1.2:1, or within any of the other ranges mentioned above in relation to the first precursor solution.

After the feeding step comes the heating step. The heating step is performed largely as described above in relation to the heating of the first precursor mixture, but this time the nanoparticle, or seed, mixture, now containing the added second precursor mixture, is heated to achieve seeded growth of the multi-core magnetic metal oxide nanoparticles in the nanoparticle mixture via thermal decomposition of the second metal precursor. The nanoparticle mixture is therefore heated to an elevated temperature sufficient to achieve growth of the nanoclusters. In other words, the nanoparticle mixture is heated to a temperature sufficient to decompose the metal precursor to achieve seeded growth of the multi-core magnetic metal oxide nanoparticles. The nanoparticle mixture may, for example, be heated to a temperature of at least about 280° C. This elevated temperature may be referred to as the second reaction temperature. The precursor mixture may be heated to at least the second reaction temperature, for example held at the second reaction temperature, for a certain period of time, known as the second reaction time. The second reaction time may be at least about 20 minutes, or at least about 30 minutes. For example, the second reaction time may be about 30 minutes. As described above in relation to the heating of the first precursor mixture, the nanoparticle mixture may be heated to the second reaction temperature in a two-step process comprising a first heating step of heating to a temperature sufficient to substantially dissolve the second metal precursor and, if present, nanoparticle clustering agent in the solvents (e.g. about 180° C.), a holding step, in which the temperature is held at this temperature for a time sufficient to substantially dissolve the metal precursor and nanoparticle clustering agent (e.g. about 30 mins), and then a second heating step, in which the temperature of the nanoparticle mixture is raised to the second reaction temperature. In general, the temperatures, reaction times, and heating procedures, may be substantially the same, or identical to those described in relation to the heating of the first precursor solution above. Therefore, the second reaction temperature/time may be the same as the first reaction temperature/time, for example.

Following the heating step, the resulting nanoparticle solution is cooled sufficiently to substantially prevent further thermal decomposition of the second metal precursor and to substantially prevent further growth of the nanoparticles, in other words to substantially halt the seeded growth of the nanoparticles. The temperature of the nanoparticle solution is therefore reduced to a temperature insufficient to cause thermal decomposition of the second metal precursor. In other words, the temperature of the nanoparticle solution is reduced sufficiently to quench growth of the nanoparticles or to substantially prevent further growth of the nanoparticles. The nanoparticle solution may, for example, be reduced to ambient (e.g. room) temperature, such as to about 25° C.

The method may then involve performing one or more further seeded growth steps 110 in which the nanoclusters are grown in size further, further enhancing the heating ability of the nanoclusters. Each further seeded growth step may be as described above in relation to the first seeded growth step, and each comprises a further feeding step and a further heating step.

In the further feeding step of each of the further seeded growth steps a further precursor mixture 112 (e.g. a further precursor solution) is added to the nanoparticle/seed mixture. The further precursor mixture comprises a further metal precursor and a further solvent. The further precursor mixture may also comprise a further nanoparticle clustering agent and/or a further reducing agent. However, it is not necessary for these additional components to be present, and the amount of nanoparticle clustering agent remaining from the first and second precursor solutions may be sufficient to achieve clustering of the nanoparticles in the further seeded growth steps without the need to add further nanoparticle clustering agent in the further precursor solution.

The components of the further precursor mixture may be as described above in relation to the equivalent components in the second precursor mixture. For example, the further solvent may be a polyol solvent, the further metal precursor may be iron(III) acetylacetonate, and the further nanoparticle clustering agent, if present, may be PAA. The corresponding components of the first/second and further precursor mixtures may be the same. For example, the first and further metal-containing precursors may be the same, the first and further solvents may be the same, and the first and further nanoparticle clustering agents may be the same. In such cases, it may be said that the first precursor mixture comprises first amounts of a metal-containing precursor/solvent/nanoparticle clustering agent and that the further precursor mixture comprises further amounts of the metal-containing precursor/solvent/nanoparticle clustering agent. Alternatively, it may be said that the first precursor mixture comprises a first precursor/solvent/nanoparticle clustering agent and that the further precursor mixture comprises the (same) metal-containing precursor/solvent/nanoparticle clustering agent. The further precursor mixture may, for example, have the same composition as the first precursor mixture, such that the chemical identity of each of the components is the same, and the relative amounts of each of the components in the further precursor mixture are the same as in the first precursor mixture. If this is the case, it could then be said that the first precursor mixture is a first amount of a precursor mixture, and that the further precursor mixture is a further amount of the precursor mixture. It is not necessary for the further precursor mixture to have the same composition as the first precursor mixture, but typically the chemical identities of the components in the precursor mixtures will be the same. For example, the further metal precursor will typically be the same as the first (and second) metal precursor (i.e. first/second and further metal precursors will be the same compound, such as iron(III) acetylacetonate), the further solvent will typically be the same as the first solvent, and, if present, the further nanoparticle clustering agent will typically be the same as the first nanoparticle clustering agent. If a separate reducing agent is present, the further reducing agent will typically be the same as the first reducing agent. In general, the further precursor solution may be prepared as described above in relation to the first/second precursor solutions.

After the feeding step comes the heating step. The heating step is performed as described above in relation to the heating of the first precursor mixture, but this time the nanoparticle, or seed, mixture, now containing the added further precursor mixture, is heated to achieve seeded growth of the multi-core magnetic metal oxide nanoparticles in the nanoparticle mixture via thermal decomposition of the further metal precursor. The nanoparticle mixture is therefore heated to an elevated temperature sufficient to achieve growth of the nanoclusters. In other words, the nanoparticle mixture is heated to a temperature sufficient to decompose the further metal precursor to achieve seeded growth of the multi-core magnetic metal oxide nanoparticles. The nanoparticle mixture may, for example, be heated to a temperature of at least about 280° C. This elevated temperature may be referred to as the further reaction temperature. The precursor mixture may be heated to at least the further reaction temperature, for example held at the further reaction temperature, for a certain period of time, known as the further reaction time. The further reaction time may be at least about 20 minutes, or at least about 30 minutes. For example, the further reaction time may be about 30 minutes. Again, the nanoparticle mixture may be heated to the further reaction temperature in a two-step process comprising a first heating step of heating to a temperature sufficient to substantially dissolve the further metal precursor and, if present, nanoparticle clustering agent in the solvent(s) (e.g. about 180° C.), a holding step, in which the temperature is held at this temperature for a time sufficient to substantially dissolve the metal precursor and nanoparticle clustering agent (e.g. about 30 mins), and then a further heating step, in which the temperature of the nanoparticle mixture is raised to the further reaction temperature. In general, the temperatures, reaction times, and heating procedures, may be substantially the same, or identical to those described in relation to the heating of the first precursor solution above. Therefore, the further reaction temperature/time may be the same as the first reaction temperature/time, for example.

Following the heating step, the resulting nanoparticle solution is cooled sufficiently to substantially prevent further thermal decomposition of the further metal precursor and to substantially prevent further growth of the nanoparticles, in other words to substantially halt the seeded growth of the nanoparticles. The temperature of the nanoparticle solution is therefore reduced to a temperature insufficient to cause thermal decomposition of the further metal precursor. In other words, the temperature of the nanoparticle solution is reduced sufficiently to quench growth of the nanoparticles or to substantially prevent further growth of the nanoparticles. The nanoparticle solution may, for example, be reduced to ambient (e.g. room) temperature, such as to about 25° C.

### Ligand Exchange

Although the nanoclusters synthesised by the seeded growth method are typically already dispersible in water, particularly when the method is carried out in a polar solvent such as a polyol solvent, their colloidal stability in aqueous solution may be improved by exchanging the polar solvent ligands (e.g. polyol ligands) on their surface with stabilising ligands capable of enhancing the solvation of the nanoparticles in aqueous media. Such ligands may be referred to as aqueous solvation stabilisation ligands, or colloidal stabilisation ligands. For example, the polar solvent ligands may be exchanged with charged (ionic) ligands with a high hydrophilicity. Preferably, the stabilising ligands are biocompatible, such as STPP. Such a ligand exchange step is made easier when polyol solvents are used in the synthesis of the nanoclusters as the resulting nanoclusters are already soluble in aqueous media and the polyol ligands can be readily exchanged.

The ligand exchange step may comprise adding a stabilising ligand, e.g. STPP, to the nanoparticle mixture, preferably in the form of an aqueous solution of the stabilising ligand. The ligand exchange step may then comprise, preferably after a sufficient time has elapsed to achieve ligand exchange (i.e. exchange of the nanoparticle surface ligands provided by the solvent with the stabilising ligands), purifying the nanoclusters to remove the polar solvent and any unreacted metal precursor, for example by dialysis against water, to produce an aqueous solution of the nanoclusters with the new surface ligands.

### Doped Nanoclusters

As mentioned above, the magnetic metal oxide nanoparticles synthesised by the method of the invention may be doped iron oxide nanoparticles. In particular they may be copper and/or zinc-doped IONPs, which have been found to exhibit particularly exceptional magnetic heating properties.

The same general method, as described above, may be used to form these doped IONPs. In order to achieve doping of the IONPs, each of the precursor mixtures (e.g. the first and second precursor mixtures) may additionally comprise a dopant-containing precursor. For example, the first precursor mixture may comprise a first dopant-containing precursor, the second precursor mixture may comprise a second dopant-containing precursor, and any further precursor mixtures may comprise a respective further dopant-containing precursor. The dopant-containing precursor in each of the precursor mixtures is generally the same dopant-containing precursor, in other words is the same substance, but this is not essential and a different dopant-containing precursor could be present in each precursor mixture. Therefore, the first and second dopant-containing precursors (and any other dopant-containing precursors that are used in the method) may be the same or different.

The dopant-containing precursor comprises a dopant, typically in the form of dopant ions. The dopant-containing precursor may for example comprise a salt of the dopant or a complex of the dopant, such as a copper or zinc salt or a copper or zinc complex. The dopant-containing precursor may for example be or comprise copper (II) acetylacetonate (Cu(acac)₂) or zinc (II) acetylacetonate (Zn(acac)₂). Other suitable dopant-containing precursors include chlorides and acetates of the dopant (e.g. CuCl₂, Cu(C₂H₃O₂)₂, ZnCl₂, or Zn(C₂H₃O₂)₂). In particular, acetates and acetylacetonates may be used as the dopant-containing precursor. The dopant-containing precursor supplies the dopant, typically in the form of dopant ions, that are incorporated into the IONPs. The precursor solutions may therefore be said to comprise dopant ions, e.g. Cu or Zn ions, provided by the dopant-containing precursor.

When a precursor mixture comprises a dopant-containing precursor, the iron-containing precursor and the dopant-containing precursor may be present in the precursor mixtures in amounts such that the amount by weight of the dopant present in the precursor mixture is less than 20% of the combined amount by weight of iron and the dopant present in the precursor mixture, for example less than 10%. For example, the amount by weight of the dopant in the precursor mixture may be about 5% of the amount by weight of iron in the precursor mixture. If more than one dopant is included in the precursor mixture, for example if the precursor mixture comprises two or more dopant-containing precursors, the combined total amount by weight of the dopants in the precursor mixture may be less than 20%, for example less than 10%, of the combined total amount by weight of iron and the dopants present in the precursor mixture.

Although doped IONPs may be formed according to the method of the invention, they may also, in another aspect of this disclosure, be formed by a one-step method, i.e. without performing any seeded growth steps. The inventors have discovered that doped IONPs provide a high magnetic heating ability without the need to perform the seeded growth steps, and the performing of the seeded growth steps is therefore not essential for yielding IONPs with a high magnetic heating ability. However, the seeded growth steps increase the ILP values of doped IONPs particularly well, and it is therefore advantageous to combine doping and seeded growth to achieve exceptional magnetic heating properties.

### Nanoclusters

The multi-core magnetic nanoparticles, or nanoclusters, formed by the method of the invention have been shown to have exceptional magnetic heating abilities when subjected to an applied alternating magnetic field. The multi-core magnetic nanoparticles synthesised by the method of the invention are therefore particularly well-suited to use in MIH applications. The nanoparticles of the invention, or compositions comprising such nanoparticles, are therefore suitable for use in magnetic hyperthermia therapy. For example, the nanoparticles or compositions comprising the nanoparticles may be used in the treatment of cancer by magnetic hyperthermia therapy.

The heating ability of nanoparticles may be quantified by the specific absorption rate (SAR), which is the power dissipated (i.e. absorbed) by the magnetic nanoparticles in an alternating magnetic field. The SAR may be determined from the initial gradient of the temperature curve obtained upon heating the mixture according to the following equation:

$$SAR = {c}_{mix} \cdot \frac{Δ T}{Δ t} \cdot \frac{{m}_{mix}}{{m}_{M}}$$

where Cₘᵢₓ is the specific heat capacity of the nanoparticle mixture (which may be approximated as the solvent's specific heat capacity), mₘᵢₓ is the total mass of the mixture, mm is the total mass of the metal component of the metal oxide of the magnetic metal oxide nanoparticles in the mixture (e.g. the total mass (m_{Fe}) of the Fe component of the nanoparticles in the mixture for IONPs), T is the temperature of the mixture, and t is time. The mass of metal component of the metal oxide (e.g. Fe) is preferably measured by inductively coupled plasma atomic emission spectroscopy (ICP-AES). The gradient or slope of the temperature curve (ΔT/dt) may be determined using the initial slope method, for example via a linear fit through an initial period (e.g. 20 seconds) of the heating profile T(t) of the nanoparticle mixture after first applying the alternating magnetic field.

Since SAR values depend on the field strength (H) and frequency (f) of the applied magnetic field, the intrinsic loss power (ILP) may be calculated to provide standardised values according to the following equation:

$$ILP = \frac{SAR}{f \cdot {H}^{2}}$$

where the SAR is measured in W/kg_{M} (where kg_{M} is the mass in kg of the metal component of the metal oxide of the metal oxide nanoparticles), the frequency (f) is measured in kHz, and the field strength (H) is measured in kA/m. The ILP values may therefore quoted in units of nH m²/kg_{M}. The ILP therefore provides standardised values for heating ability that can be readily compared.

The heating profile, and the heating ability of the nanoparticles, may be measured using a calorimetric analyser. Preferably, the heating ability is measured under adiabatic or pseudo-adiabatic conditions. In particular, the heating profile, T(t), of the nanoparticle mixture is preferably measured under adiabatic or pseudo-adiabatic conditions. The heating ability of the nanoparticles is preferably measured in a solvent in which the nanoparticles are colloidally stable such that agglomeration of the nanoparticles is avoided. For example, the heating ability may be measured in a polar solvent, in particular in a polyol solvent such as triethylene glycol. The heating ability of the nanoparticles may be measured with the nanoparticles initially at a temperature of 25° C. before the magnetic field is applied.

The nanoclusters synthesised by the method of the invention generally have an ILP of at least about 6 nH m²/kg_{M}. For example, nanoclusters synthesised using a single seeded growth step may have an ILP of at least 6 nH m²/kg_{M}. The nanoclusters synthesised by the method of the invention may have higher ILP values of at least about 7 nH m²/kg_{M}, and preferably have an ILP of at least about 8 nH m²/kg_{M}. For example, nanoclusters synthesised using two (i.e. a first and a second) seeded growth step may have an ILP of at least about 7 nH m²/kg_{M}, and preferably have an ILP of at least about 8 nH m²/kg_{M}. Doped IONPs synthesised by the method of the invention may have still higher ILP values, particularly those doped with copper and/or zinc. For example, doped IONPs synthesised by the method of the invention may have an intrinsic loss parameter of at least about 10 nH m² kg_{M}⁻¹, or even at least about 12 nH m² kg_{M}⁻¹.

As mentioned above, the multi-core magnetic nanoparticles synthesised by the seeded growth method of the invention comprise a plurality of individual cores or grains, which are bound together to form the multi-core nanoparticles, or nanoclusters. It has been found that the cores of the nanoclusters are generally monocrystalline structures. In other words, each of the cores typically consists of a single crystal. The nanoclusters may therefore be said to comprise, or consist essentially of, monocrystalline cores.

In each seeded growth step the nanoclusters increase in size as more single-crystal cores are added to the nanoclusters. This means that the overall size of the nanoclusters increases with each seeded growth step, while the individual grain size remains relatively constant. The nanoclusters synthesised by the invention may have an average diameter of at least about 25 nm, and preferably at least about 30 nm. The individual cores may have an average diameter of less than about 15 nm.

The quoted nanocluster overall diameters are measured by transmission electron microscopy (TEM), for example from an analysis of TEM image data, and are calculated as a number average. The diameters may therefore be the mean (number average) diameter as measured from one or more TEM images of the nanoparticles. For example, the diameters of a plurality of nanoparticles (e.g. at least 200 nanoparticles) may be measured (e.g. using image processing software such as ImageJ) and the average (mean) value calculated. The diameter may be calculated (for non-spherical nanoparticles, for example) as the average of the maximum diameter and the diameter perpendicular (in the image plane) to this maximum diameter.

The quoted core diameters are measured by X-ray diffraction (XRD), which measures the crystallite diameter and is therefore a good approximation of the actual core diameter of monocrystalline cores. The core diameters quoted may therefore be referred to alternatively as the crystallite diameters of the nanoparticle cores. The X-ray diffraction diameter (D_{XRD}) is determined using the well-known Scherrer equation, preferably by evaluating the most intense diffraction peak. More specifically, the crystallite diameter (D_{XRD}) may be evaluated from the peak width at full-width-half-maximum (FWHM) intensity of the most intense diffraction peak (e.g. of the {311} planes, diffracted at 20=41° for IONPs) using the Scherrer equation:

$${D}_{XRD} = \frac{K λ}{βcosθ}$$

where K is the dimensionless shape factor, λ is the X-ray wavelength, β is the peak width at FWHM in radians, and θ is the Bragg angle. The quoted core diameters are therefore the core diameters (or crystallite diameters of the cores) as measured by XRD, specifically using the Scherrer equation.

The magnetic nanoclusters synthesised by the seeded growth method are superparamagnetic. The nanoclusters retain their superparamagnetic properties due to the size of the individual grains remaining below the superparamagnetic limit, even though the overall size of the nanoclusters themselves is much larger. This avoids further agglomeration, and hence precipitation, of the nanoparticles following their formation. The individual cores may therefore have an average diameter below the superparamagnetic limit.

The combination of the high magnetic moment of the nanoclusters together with their superparamagnetic properties makes them particularly well-suited to use in magnetic particle imaging (MPI) applications, which requires this combination of properties to achieve a strong signal. Indeed, nanoclusters synthesised according to the method of the invention have been shown to give a strong MPI signal when subjected to magnetic fields typical employed in MPI applications, and the strength of the MPI signal has also been found to have a linear relationship with the concentration of the nanoclusters. The nanoclusters of the invention, or compositions comprising such nanoclusters, may therefore be used in MPI.

In particular, the nanoclusters of the invention may be used in combination MPI/MIH applications in which the nanoclusters are used for MIH therapy while simultaneously being imaged using MPI. MPI can be used to determine the local concentration of the nanoparticles in vivo at the site undergoing MIH therapy, and combining MPI with MIH therefore allows for the strength of the applied magnetic fields used in the MIH therapy to be tailored to the local concentration of the nanoclusters for maximum effectiveness. The combination of properties of the nanoclusters of the invention, namely their high heating ability, highly magnetic properties, and superparamagnetism makes them very well suited to such combination MIH/MPI applications.

### EXAMPLES

### Methods

### Reference Example 1

IONPs were produced via thermal decomposition of a metal precursor in a polyol solvent. A precursor solution was prepared by dispersing iron(III) acetylacetonate (Fe(acac)₃) in TREG, which dissolved when the temperature exceeded 80° C. to yield a 0.13 M Fe(acac)₃ precursor solution. The precursor solution was heated from room temperature to 180° C. at a rate of 3° C./min and held at this temperature for 30 minutes prior to thermal decomposition of the Fe(acac)₃. The temperature was then increased to 280° C. at a rate of 5° C./min and the reaction mixture (i.e. the heated precursor solution) was held at this temperature for reaction times of 1 hour, 6 hours or 24 hours to yield nanoparticle solutions comprising iron oxide nanoparticles via thermal decomposition of the Fe(acac)₃ before cooling to room temperature. The solution was magnetically stirred (500 rpm) throughout the synthesis.

### Reference Example 2

IONPs were produced via thermal decomposition of a metal precursor in a polyol solvent. A precursor solution was prepared by dispersing iron(III) acetylacetonate (Fe(acac)₃) in TREG, which dissolved when the temperature exceeded 80° C. to yield a 0.07 M Fe(acac)₃ precursor solution. PAA was also added to the precursor solution in varying amounts to yield precursor solutions having PAA concentrations of 20, 33, 66 and 100 mM, depending on the amount of PAA added. The precursor solution was heated from room temperature to 180° C. at a rate of 3° C./min and held at this temperature for 30 minutes prior to thermal decomposition of the Fe(acac)₃. The temperature was then increased to 280° C. at a rate of 5° C./min and the reaction mixture was held at this temperature for a reaction time of 30 minutes to yield nanoparticle solutions comprising iron oxide nanoparticles via thermal decomposition of the Fe(acac)₃ before cooling to room temperature. The solution was magnetically stirred (500 rpm) throughout the synthesis.

### Example 1

### Producing a Nanoparticle Solution

IONPs were produced via thermal decomposition of a metal precursor in a polyol solvent. A precursor solution was prepared by dispersing iron(III) acetylacetonate (Fe(acac)₃) in TREG, which dissolved when the temperature exceeded 80° C. to yield a 0.07 M Fe(acac)₃ precursor solution. PAA was also added to the precursor solution to yield a precursor solution having a PAA concentration of 66 mM. The precursor solution was heated from room temperature to 180° C. at a rate of 3° C./min and held at this temperature for 30 minutes prior to thermal decomposition of the Fe(acac)₃. The temperature was then increased to 280° C. at a rate of 5° C./min and the reaction mixture was held at this temperature for a reaction time of 30 minutes to yield a nanoparticle seed solution comprising iron oxide nanoparticles via thermal decomposition of the Fe(acac)₃ before cooling to room temperature. The solution was magnetically stirred (500 rpm) throughout.

### Seeded Growth Steps

A first seeded growth step was then performed as follows. A selected volume of additional precursor solution (10 ml) was added at room temperature to the previously synthesised nanoparticle solution. The additional precursor solution was the same composition as the initial precursor solution used to produce the nanoparticle solution. The Fe molarity therefore remained constant in all precursor solutions, before and during the seeded growth. The nanoparticle solution, now with added precursor solution, was magnetically stirred for 5 min and then heated to 280° C. at a rate of 5° C./min. The reaction mixture (i.e. the nanoparticle solution) was held at this temperature for a reaction time of 30 minutes to achieve seeded growth of the nanoparticles via thermal decomposition of the Fe(acac)₃ before cooling to room temperature to terminate nanoparticle growth. The solution was magnetically stirred (500 rpm) throughout.

A second seeded growth step was then performed in the same way as described above, with a further additional volume of the precursor solution (10 ml) being added to the cooled nanoparticle solution yielded by the first seeded growth step. The nanoparticle solution was magnetically stirred for 5 min and then heated to 280° C. at a rate of 5° C./min. The reaction mixture (i.e. the nanoparticle solution) was held at this temperature for a reaction time of 30 minutes to achieve seeded growth of the nanoparticles via thermal decomposition of the Fe(acac)₃ before cooling to room temperature to terminate nanoparticle growth. The solution was magnetically stirred (500 rpm) throughout.

### Repetitions

The above procedure (producing a nanoparticle solution followed by two seeded growth steps) was performed separately three times to provide three nanoparticle solutions for analysis.

### Example 2

Although the IONPs synthesised in Example 1 were stable in the polyol solution in which they were synthesised, and were dispersible in water, their colloidal stability in aqueous solution was improved by exchanging the polyol ligands with a ligand capable of enhancing the colloidal stability of the IONPs in aqueous solution. To achieve long-term colloidal stability in water, the polyol ligands were exchanged with a stabiliser, namely STPP, which is a biocompatible stabiliser. To secure the attachment of STPP on the IONPs, 200 ml of a 2.5 mM aqueous STPP solution were added under rigorous stirring to 30 ml of the IONP polyol solution formed following the seeded growth steps of Example 1. The resulting IONP solution (13% by volume polyol, 87% by volume water) was kept overnight under moderate stirring to ensure successful ligand exchange of the polyol (TREG) with STPP. To remove excess STPP, unreacted precursor, and the polyol, the STPP-capped IONPs were purified by dialysis against deionised water in a cellulose acetate dialysis bag (Spectra/Por™ 12000-14000 g/mol MWCO, Standard RC Trial Kit) for 72 hours at room temperature, during which the water was replaced frequently.

### Example 3a

Copper-doped IONPs were produced via thermal decomposition of a metal precursor in a polyol solvent. The synthesis was performed in the same way as in Example 1, with the addition of copper(II) acetylacetonate (Cu(acac)₂) to the precursor solution so that the amount of copper in the precursor solution was 5% by weight of the total amount by weight of copper and iron in the precursor solution.

### Example 3b

Zinc-doped IONPs were produced via thermal decomposition of a metal precursor in a polyol solvent. The synthesis was performed in the same way as in Example 1, with the addition of zinc(II) acetylacetonate (Zn(acac)₂) to the precursor solution so that the amount of zinc in the precursor solution was 5% by weight of the total amount by weight of zinc and iron in the precursor solution.

### Characterisation

The nanoparticles synthesised in the examples were characterised as per the following methods.

IONPs that did not undergo ligand exchange were precipitated with ethyl acetate (2:1 ratio by volume of ethyl acetate to sample), magnetically decanted and washed with excess ethanol in triplicate before analysis.

TEM images were captured using a JEOL 1200 EX microscope at a 120 kV acceleration voltage. IONP dispersions (after redispersion in deionised water) were drop-casted on carbon-coated copper grids and air-dried at room temperature. Particle size analysis from TEM images was performed manually using the image analysis software ImageJ. For non-spherical particles, the particle diameter was obtained by averaging the maximum diameter and the diameter at a right angle to this maximum diameter. For each sample >200 particles were measured to determine the average diameter and standard deviation (D_{TEM}±σ_{TEM}). Aberration-corrected high-resolution TEM (HRTEM) was performed in a Titan Themis 60-300 equipped with an image corrector, probe corrector, and monochromator at 200 kV.

XRD patterns of dried samples were obtained using a PANalytical X'Pert3 (Malvern) diffractometer equipped with a CoK_{α} radiation source (λ=1.79 Å) operated at 40 mA. The crystallite diameter (D_{XRD}) was evaluated from the peak width at full-width-half-maximum (FWHM) intensity of the most intense diffraction peak, i.e., of the {311} planes, diffracted at 2θ=41° using the Scherrer equation:

$${D}_{XRD} = 0.89 \cdot λ / FWHM \cdot cos ( θ ) .$$

Fourier transformation infrared (FTIR) spectra were recorded using an attenuated total reflectance probe (Spectrum 100 FTIR, Perkin-Elmer).

The hydrodynamic diameter (Dₕ) was determined by DLS. DLS was performed for ligand exchanged IONPs with a DelsaMax-Pro (Beckman Coulter) at room temperature (e.g. 22° C.). IONP solutions were diluted with deionised water until the hydrodynamic diameter (Dₕ) obtained plateaued (typically after a four-fold dilution).

The concentration of Fe in the IONPs (C_{Fe-IONP}) was identified by ICP-AES. First, the samples were washed (as described above, i.e. with excess ethanol in triplicate) and weighed before dissolution in concentrated nitric acid at 60° C. Thereafter, these solutions were diluted with deionised water to obtain a 2% by weight (wt./wt.) nitric acid solution (the same as the standards used for calibration) for Fe quantification using a Varian 720 ICP-AES (Agilent) spectrometer.

The IONP magnetic properties were characterised by acquiring M-H plots (where M is the mass magnetisation (emu/g_{Fe}) and H is the applied field strength (Oe), with M on the y-axis and H on the x-axis) at 5 and 300 K with applied fields up to 50 kOe and zero-field-cooled (ZFC) and field-cooled (FC) magnetisation versus temperature measurements (from 300-5 K) in 100 Oe, obtained with an MPMS superconducting quantum interference device magnetometer (Quantum Design).

The particles' heating abilities in an alternating magnetic field were evaluated with a calorimetric analyser (G2 driver D5 series, nB nanoScale Biomagnetics) at a frequency (f) of 488 kHz and a field strength (H) of 308 Oe (=25 kA/m). The temperature was recorded with a GaAs-based fibre optic probe immersed in a vial containing approximately 1 ml of the IONP solution being measured. A sealed glass (Dewar flask at <0.1 Pa) provided thermal insulation of the sample vial, rendering it a pseudo-adiabatic system. The particles' specific abortion rate (SAR), i.e., the power dissipated by the magnetic particles, was obtained by the following equation:

$$SAR = {c}_{sol} \cdot \frac{Δ T}{Δ t} \cdot \frac{{m}_{sol}}{{m}_{Fe}}$$

where cₛₒₗ is the specific heat capacity of the IONP solution (approximated as the solvent's specific heat capacity), mₛₒₗ is the total mass of the solution, m_{Fe} is the total mass of the Fe component of the IONPs in the solution, T is the temperature of the solution, and t is time. ΔT/dt was determined using the initial slope method via a linear fit through the first 20 s of the heating profile T(t) after first applying the alternating magnetic field.

The intrinsic loss power (ILP) was determined according to the following equation to provide standardised values:

$$ILP = \frac{SAR [ W / {kg}_{Fe} ]}{f [ kHz ] \cdot {H}^{2} [ {( kA / m )}^{2} ]}$$

### Results

### Reference Example 1

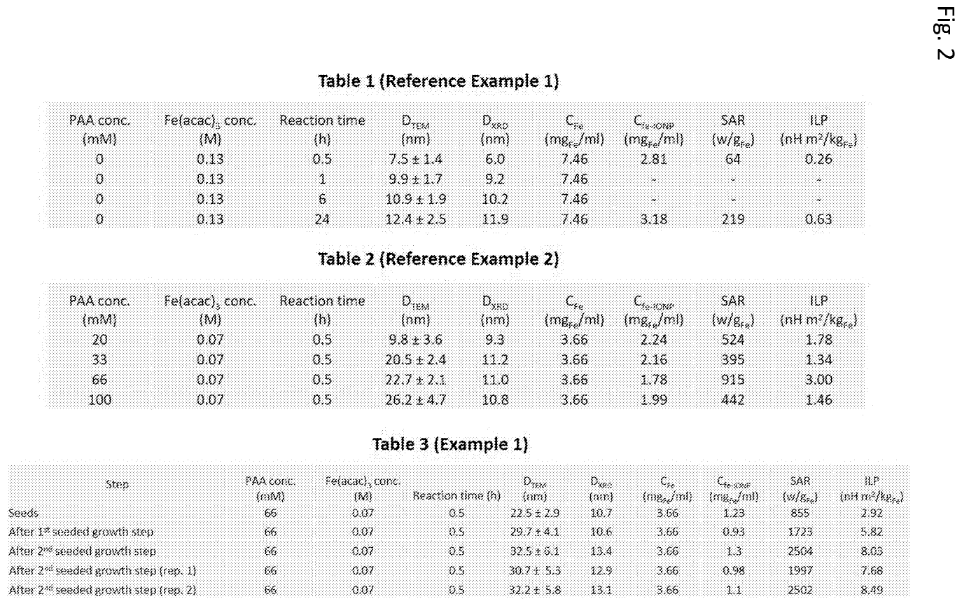

Table 1 in FIG. 2 summarises the properties of the IONPs synthesised according to Reference Example 1. The IONPs increased in size with increasing reaction time. For the shortest reaction time (30 min) particles having a diameter as measured by TEM (D_{TEM}) of 7.5±1.4 nm were obtained. Increasing the reaction time to 1 h, 6 h and 24 h yielded IONPs having a D_{TEM} of 9.9±1.7 nm, 10.9±1.9 nm and 12.4±2.5 nm, respectively.

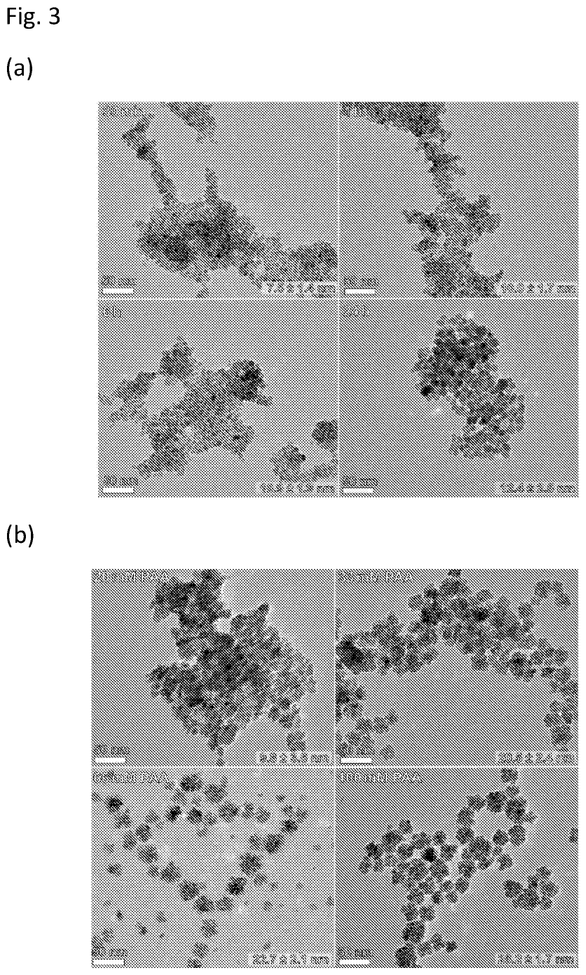

XRD analysis confirmed that all reaction times resulted in IONPs of inverse spinel structure (i.e., the most magnetic form; magnetite (Fe₃O₄) or maghemite (γ-Fe₂O₃)). The crystallite size (the XRD measured diameter, D_{XRD}) increased likewise with the reaction time from D_{XRD}=6.0 nm for 30 min to D_{XRD}=9.2, 10.2 and 11.9 nm for 1 h, 6 h and 24 h, respectively. The comparable diameters obtained from XRD and TEM indicate that IONPs were monocrystalline, single core IONPs, which was also confirmed by TEM, as can be seen from FIG. 3(a), which clearly shows that the IONPs were not clustered. The solubility of IONPs in the polyol solution was observed to be low.

The IONP heating ability increased with the reaction time (i.e. with the IONP size) from negligible heating performance for 30 min (SAR=64 W/g_{Fe}) to an SAR of 219 W/g_{Fe} for the 24 h synthesis. However, such long reaction times are not practical in high-scale production is required, and the heating ability remained low even for long reaction times, with an ILP of only 0.63 nH m²/kg_{F}e even for the 24 h reaction time. In summary, all reaction times yielded small IONPs with relatively poor heating ability.

### Reference Example 2

When the synthesis was carried out in the presence of PAA, as per Reference Example 2, TEM analysis showed that the IONPs were clustered. The TEM images shown in FIG. 3(b) clearly show that the IONPs grains are clustered together to form multi-core (i.e. clustered) nanoparticles.

Table 2 in FIG. 2 summarises the properties of the IONPs synthesised according to Reference Example 2. IONP aggregation was observed at 20 mM PAA, yielding highly polydisperse IONP clusters (D_{TEM}=9.8±3.6 nm). When increasing the amount of PAA to 33 mM, 66 mM, and 100 mM well-defined and monodisperse clustered nanoparticles were obtained with average diameters (D_{TEM}) of 20.5 nm, 22.7 nm and 26.2 nm, respectively.

XRD analysis confirmed that all IONPs possessed the inverse spinel structure with core crystallite diameters (D_{XRD}) of 9.3 nm, 11.2 nm, 11 nm and 10.8 nm for 20, 33, 66, and 100 mM PAA, respectively. TEM and XRD analysis therefore showed that while an increase in PAA concentration resulted in larger IONP clusters, the crystallite size remained relatively constant. This confirms the multicore nature of IONPs synthesised with PAA.

The heating abilities of the single core and clustered IONPs in an alternating magnetic field shows a clear enhancement of the SAR for clustered IONPs. The SAR was enhanced with increasing PAA concentrations reaching 915 W/g_{Fe} (ILP=3.0 nH m²/kg_{Fe}) for IONPs synthesised with 66 mM PAA. The increase in heating efficiency is attributed to the structural-magnetic properties of the NFs, which are composed of highly ordered nanocrystals that do not behave like isolated grains.

When increasing the PAA concentration to 100 mM the SAR dropped (despite an increase in NF size) to 442 W/g_{Fe} (ILP=1.46 nH m²/kg_{Fe}) indicating that higher PAA concentration deteriorates the magnetic coupling. In addition, when using 100 nM PAA the solution became highly viscous at room temperature, which hampered the ligand exchange step.

Magnetic hysteresis curves at 300 K showed that the IONPs synthesised using PAA concentrations of 20, 33, and 66 mM PAA were superparamagnetic. Their coercivities were less than 50 Oe (20, 18.3, and 17 Oe, respectively) and they had a mass magnetization (M) of 69.9, 100 and 105 emu/g_{Fe} respectively.

### Example 1

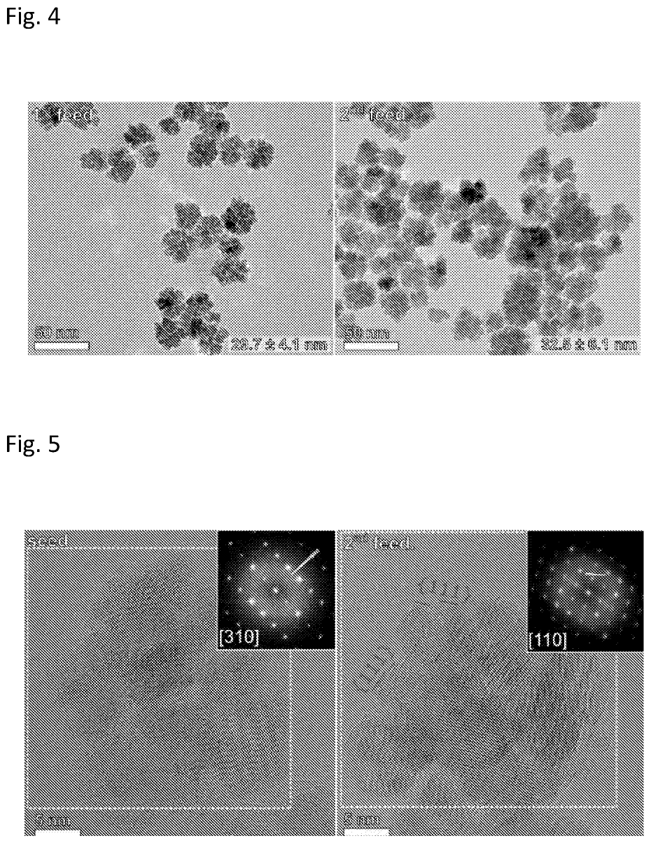

Table 3 in FIG. 2 summarises the properties of the IONPs synthesised according to Example 1. TEM analysis showed that the IONP size increased with each feeding step. The diameter of the IONPs increased from D_{TEM}=22.5±2.9 nm for the seed IONPs in the initial nanoparticle solution to 29.7±4.1 nm after the first seeded growth step, and to D_{TEM}=32.5±6.1 after the second seeded growth step. On the other hand, XRD measurements revealed that the core or grain size increased only slightly from D_{XRD}=10.7 nm for the initial seed nanoparticles to D_{XRD}=10.6 nm after the first seeded growth step, and to D_{XRD}=13.4 nm after the second seeded growth step. This demonstrates that the nanoparticles primarily grow in size due to the addition of further individual cores or grains in each seeded growth step. TEM images also confirmed the multi-core nature of the nanoparticles, as shown in FIG. 4.

The heating ability increased significantly with each seeded growth step, doubling after the first (SAR=1723 W/g_{Fe}), and almost tripling after the second seeded growth step to SAR=2504 W/g_{F}e, i.e. an ILP=8.03 nH m²/kg_{Fe} during the first run. In the first repetition of the procedure an ILP of 7.68 nH m²/kg_{Fe} was obtained after the second seeded growth step, and in the second repetition of the procedure an ILP of 8.49 nH m²/kg_{Fe} was obtained after the second seeded growth step, thus demonstrating that a high ILP can be obtained consistently when performing the method of the invention.

HRTEM comparison of the IONFs seeds (i.e. before the seeded growth steps) and the grown nanoparticles after the first seeded growth step revealed that both are made up of aligned grains viewed down a single zone axis (FIG. 5). For the nanoparticle seeds, the particle was viewed down the [310] zone axis. The broadening of the spot in the Fourier transformation (FIG. 5 insert) indicates a slight lattice mismatch between the single cores and reflects a deviation in the grain orientation (arcing in the spots is shown by the white lines in the inset) of 2.2±0.6 degrees. The grown nanoparticles after the first seeded growth step are seen to be highly faceted with predominantly {111} facets exposed on their surface. The nanoparticles are viewed down a single [110] zone axis, with a deviation in the grain orientation (white lines in the inset) being 2.1° (seeds) and 3.4° (grown). This shows that the NFs are made up of substantially aligned crystalline domains deviating by a small angle, which increased as the nanoparticles grew larger.

The HRTEM, TEM and XRD studies indicated seeded growth by coalescence. The IONP growth with each feeding step occurred by aligned aggregation of newly formed single core crystals. More of these building blocks are added with each feeding step, while the fusion (or alignment) of single core crystals in the nanoparticles progresses.

### Example 2

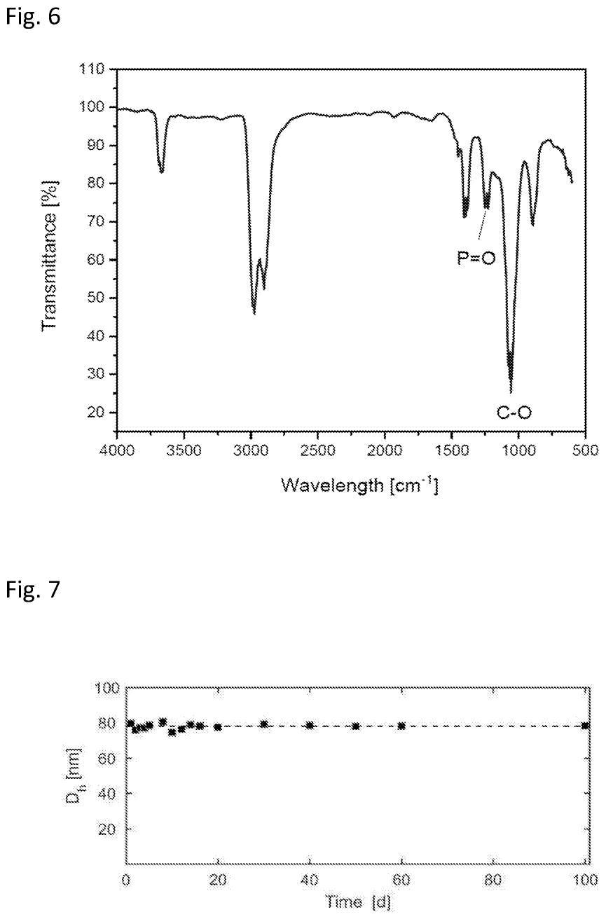

FTIR results (FIG. 6) demonstrated that STPP successfully substituted the TREG molecules as ligands on the IONPs during the ligand exchange step. After the STPP ligand exchange, the characteristic bonds of PAA disappeared, while P—O and P═O stretching bands appeared, located between 1200 and 1300 cm⁻¹ corresponding to STPP.

Replacing polyol ligands with STPP increased the electrostatic and steric repulsion significantly, which improves the nanoparticles' long-term colloidal stabilisation. The hydrodynamic diameter of STPP-capped IONPs was Dₕ=78 nm and did not show any further increase after more than three months (FIG. 7). The size of IONPs did not change substantially after dialysis (D_{TEM}=32±5.3 nm) and XRD analysis showed that the ligand exchange reaction had no effect on the crystal structure and crystallite size remained relatively unchanged at D_{XRD}=10.9 nm.

### Examples 3a and 3b

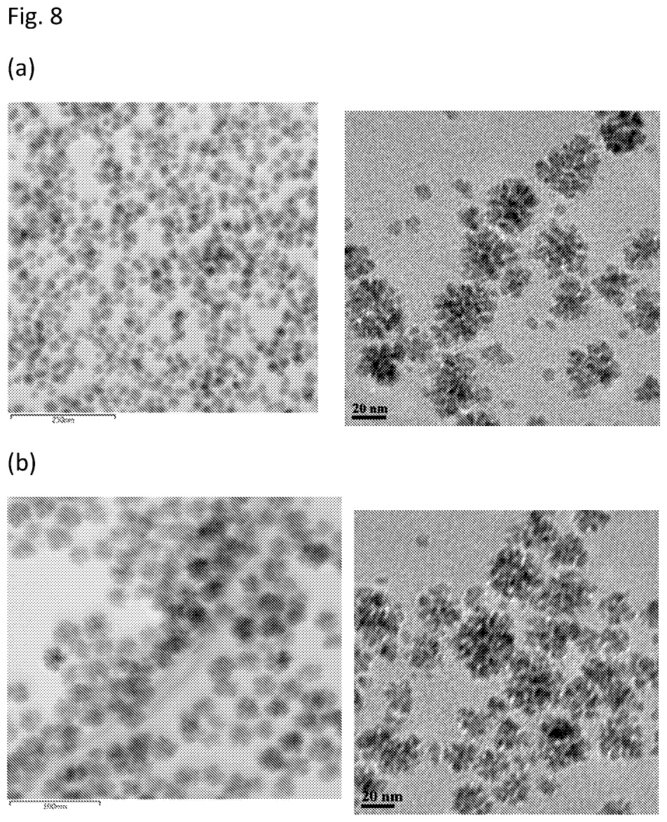

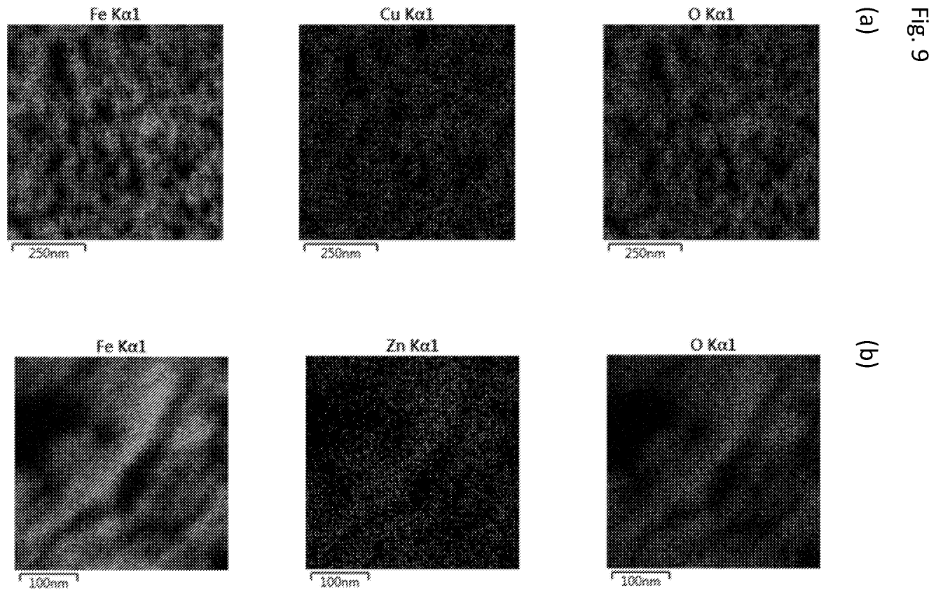

TEM images confirmed the multi-core nature of the nanoparticle seeds and after the first and second seeded growth steps (FIG. 8). TEM elemental mapping also confirmed that the copper and zinc were incorporated into the nanoparticles, as demonstrated by the spatial correlation between the distributions of iron and copper/zinc in the elemental mapping TEM images shown in FIG. 9.

High ILP values of 4.41 nH m²/kg_{Cu} and 4.42 nH m²/kg_{Zn} were calculated for the copper- and zinc-doped IONPs, respectively. The magnitude of the ILP values increased between the feed steps, with the ILP values of the copper-doped nanoparticles increasing by a factor of 1.62 between the first and second seeded growth steps, and the ILP values of the copper-doped nanoparticles increasing by a factor of 2.07 between the first and second seeded growth steps, demonstrating that the seeded growth approach leads to rapidly increasing ILP values for each feed step.

The description of the invention provided above is intended to introduce various aspects and features of the invention in a non-limiting manner. For clarity and brevity, features and aspects of the invention may be described in the context of particular embodiments. However, it should be understood that features of the invention that are described only in the context of one or more embodiments may be employed in the invention in the absence of other features of those embodiments, particularly where there is no inextricable functional interaction between those features. Even where some functional interaction between the features of an embodiment is discernible, it is to be understood that those features are not inextricably linked if the embodiment would still fulfil the requirements of the invention without one or more of those features being present. Thus, where features are, for brevity, described in the context of a single embodiment, those features may also be provided separately or in any suitable sub-combination. It should also be noted that features that are described in the context of separate aspects and embodiments of the invention may be used together and/or be interchangeable wherever possible. It is also to be understood that all disclosed optional features, values and ranges may be combined with any other optional features, values and ranges in any suitable combination and it should therefore be understood that all such combinations are therefore disclosed. For example, individual features may be extracted from a plurality of lists of features and combined, and all such combinations are to be understood to be disclosed herein. Features described in connection with the invention in different contexts (e.g. method, product) may each have corresponding features definable and/or combinable with respect to each other, and these embodiments are specifically envisaged. Where the term “comprise” is used, this should also be understood to include “consists of” or “consists essentially of” unless the context implies otherwise. Where the term “consists of” is used, this should be understood to also include “consists essentially of”.

1. A method for synthesising multi-core magnetic metal oxide nanoparticles, the method comprising:
providing a first precursor mixture comprising a first metal-containing precursor, a first solvent, and a first nanoparticle clustering agent;
heating the first precursor mixture to thermally decompose the first metal-containing precursor to produce a nanoparticle mixture comprising multi-core magnetic metal oxide nanoparticles; and
performing a first seeded growth step comprising:
a feeding step in which a second precursor mixture is added to the nanoparticle mixture, the second precursor mixture comprising a second metal-containing precursor and a second solvent; and
a heating step in which the nanoparticle mixture is heated to thermally decompose the second metal-containing precursor to achieve growth of the multi-core magnetic metal oxide nanoparticles.

2. The method of claim 1, wherein the first solvent is a first polar solvent, and/or wherein the second solvent is a second polar solvent.

3. The method of claim 1, wherein the first solvent is a first polyol solvent, and/or wherein the second solvent is a second polyol solvent.

4. The method of claim 1, wherein:
the second solvent is the same as the first solvent; and/or
the second metal-containing precursor is the same as the first metal-containing precursor.

5. The method of claim 1, wherein the second precursor mixture comprises a second nanoparticle clustering agent.

6. The method of claim 1, further comprising performing at least one further seeded growth step after the first seeded growth step, each further seeded growth step comprising:
a feeding step in which a further precursor mixture is added to the nanoparticle mixture, the further precursor mixture comprising a further metal-containing precursor and a further solvent; and
a heating step in which the nanoparticle mixture is heated to thermally decompose the further metal-containing precursor to achieve further growth of the multi-core magnetic metal oxide nanoparticles.

7. (canceled)

8. The method of claim 6, wherein more than one further seeded growth step is performed.

9. The method of claim 6, wherein:
a) in each further seeded growth step, the further solvent is a further polyol solvent;
b) the further metal-containing precursor in each further seeded growth step is the same as the first metal-containing precursor and/or the second metal-containing precursor;
c) the further solvent in each further seeded growth step is the same as the first solvent and/or the second solvent; and/or
d) each further precursor mixture comprises a further nanoparticle clustering agent.

10. (canceled)

11. (canceled)

12. The method of claim 1, wherein the nanoparticle clustering agent in any or each of the precursor mixtures is a polyelectrolyte.

13. The method of claim 1, wherein the molar ratio of the first nanoparticle clustering agent to the metal provided by the first metal-containing precursor is at least 0.6:1.

14. The method of claim 1, wherein the molar ratio of the first nanoparticle clustering agent to the metal provided by the first metal-containing precursor is in the range of 0.6:1 to 1.4:1.

15. The method of claim 1, wherein, in each of the precursor mixtures, the molar ratio of the nanoparticle clustering agent to the metal provided by the metal-containing precursor is at least 0.6:1.

16. The method of claim 1, wherein, in each of the precursor mixtures, the molar ratio of the nanoparticle clustering agent to the metal provided by the metal-containing precursor is in the range of 0.6:1 to 1.4:1.

17. The method of claim 1, wherein the metal-containing precursor in any or each of the precursor mixtures is a metal salt or a metal complex.

18. The method of claim 1, wherein the multi-core magnetic metal oxide nanoparticles are ferrite multi-core magnetic metal oxide nanoparticles.

19. (canceled)

20. The method of claim 18, wherein the metal-containing precursor in any or each of the precursor mixtures is iron(III) acetylacetonate.

21. The method of claim 18, wherein each of the precursor mixtures further comprises a respective dopant-containing precursor, and wherein the ferrite multi-core magnetic metal oxide nanoparticles are doped ferrite multi-core magnetic metal oxide nanoparticles.

22. The method of claim 21, wherein the dopant in each of the dopant-containing precursors is copper or zinc.

23. The method of claim 1, wherein the synthesised multi-core magnetic metal oxide nanoparticles have an intrinsic loss parameter of at least about 6 nH m² kg_{M}⁻¹; an average diameter of at least about 25 nm as measured by transmission electron microscopy; and/or wherein the synthesised multi-core magnetic metal oxide nanoparticles have individual core diameters of less than about 15 nm as measured by X-ray diffraction.

24. (canceled)

25. Multi-core magnetic metal oxide nanoparticles synthesised according to the method of claim 1.

26. (canceled)

27. (canceled)
